(19) United States
(12) Patent Application Publication
(10) Pub. No.: US 2024/0225044 A1
(43) Pub. Date: Jul. 11, 2024

(54) EDIBLE 3D PRINTING BIOINK AND PREPARATION METHOD THEREFOR AND APPLICATION THEREOF IN CULTIVATED MEAT

(71) Applicant: CHINA MEAT RESEARCH CENTER, Beijing (CN)

(72) Inventors: Shouwei WANG, Beijing (CN); Haijuan HU, Beijing (CN); Yingying LI, Beijing (CN); Shilei LI, Beijing (CN); Feng YANG, Beijing (CN)

(73) Assignee: CHINA MEAT RESEARCH CENTER, Beijing (CN)

(21) Appl. No.: 18/395,690

(22) Filed: Dec. 25, 2023

Related U.S. Application Data
(63) Continuation of application No. PCT/CN2023/129008, filed on Nov. 1, 2023.

(30) Foreign Application Priority Data
Jan. 6, 2023 (CN) 202310015447.5

(51) Int. Cl. A23J 3/26; A23J 3/06; B33Y 70/00; B33Y 80/00

(52) U.S. Cl. CPC A23J 3/26; A23J 3/06; B33Y 70/00; B33Y 80/00

(57) ABSTRACT

The present invention relates to an edible 3D printing bioink, a preparation method therefor and an application thereof in a cultivated meat. Raw materials of the bioink according to the present invention include pectin, glutamine transaminase, a first protein component, and a second protein component; the bioink has good biocompatibility, printability and stability, and supports three-dimensional growth of cells; and the bioink is edible without photoinitiators for 3D printing, and therefore has a broad application prospect in fields of food science, cellular agriculture and biomedicine.

### CROSS-REFERENCE TO RELATED APPLICATIONS

The application claims priority to Chinese patent application No. 202310015447.5, filed on Jan. 6, 2023, the entire contents of which are incorporated herein by reference.

### TECHNICAL FIELD

The present invention relates to the technical field of bioink materials, and in particular, to an edible 3D printing bioink, a preparation method therefor and an application thereof in a cultivated meat.

### BACKGROUND

Bio-cultivated meat is also known as cultivated meat, cell-cultivated meat, clean meat, and the like; and cells are cultured in vitro to produce lump meat that is highly similar to real muscle tissues in terms of nutrition, appearance, texture, and flavor. Conventional two-dimensional cell culture methods are unable to meet the requirements for cell growth and development into lump meat because they do not really simulate the micro-environment in vivo. Different from conventional two-dimensional culture of cells, three-dimensional culture of cells can simulate the situation of cells in vivo, so that cells present a three-dimensional growth state. Therefore, in order to construct muscle tissues with real texture in vitro, scaffold materials prepared by a bioink with good biocompatibility need to be used to realize the three-dimensional culture of cells, thereby achieving the purpose of in vitro shaping and construction of muscle tissues.

In natural muscle tissues, an extracellular matrix is part of an animal tissue and mainly consists of protein and polysaccharide. In general, the ideal 3D printing bioink should be similar to the extracellular matrix in terms of structure and function and has good biocompatibility, which can promote adhesion and three-dimensional growth of cells thereon. However, due to the poor biocompatibility of protein and polysaccharide-based materials, the cells are unable to adhere and grow directly thereon. Therefore, in the prior art, ink materials and cells are usually mixed and then directly printed into a product (for example, Patent Application CN2021116690280), instead of cells being inoculated into the bioink materials. The cells present the spatial three-dimensional growth state through adhesion, three-dimensional growth, and proliferation and autonomously develop into lump meat.

At present, methacrylate modification of proteins and polysaccharides is required to enhance the biocompatibility and printability of protein and polysaccharide materials. Gelatin is a protein with poor biocompatibility and printability, and subjected to acidation modification of methacrylonitrile to obtain gelatin methacrylamide (GelMA) with good biocompatibility and printability in practical applications. Gelatin methacrylamide (GelMA) is a photosensitive biomaterial. During printing, it can be cross-linked and cured with photoinitiators under blue light or ultraviolet light to form a three-dimensional structure with a specified strength to support the three-dimensional growth of skeletal muscle cells and is mainly applied to the fields of medicine and tissue engineering. Pectin is a macromolecule polysaccharide naturally occurring in fruits. It cannot be stabilized in a culture medium due to good solubility, and provide cells with adhesion points for growth due to poor printability. Therefore, it can be used only as a gelling agent and an emulsifier in the food industry, instead of the bioink. It has been reported that the pectin has significantly biocompatibility and printability after subjected to acidation modification of methacrylonitrile, and it can be used as an ink material to cultivate cells with photoinitiators. Unlike the pharmaceutical and tissue engineering fields, bioinks applied to bio-cultivated meat need to have edible properties. Because inedible photoinitiators are introduced during printing of the gelatin subjected to acidation modification of methacrylonitrile and the pectin bioink, they cannot be applied to the production and processing of cultivated meat, which limits the application of the above materials in the field of cultivated meat. Therefore, in order to solve the above problems, it is necessary to develop a novel edible bioink material without photoinitiators during printing, and the bioink material has good biocompatibility and can support the three-dimensional growth of cells and is used in the field of the cultivated meat.

### SUMMARY

The purpose of the present invention is to provide an edible 3D printing bioink, a preparation method therefor and an application thereof in a cultivated meat.

Compared with a method for preparing the cultivated meat by mixing cells with the bioink and performing 3D printing on the mixture, a method for preparing the cultivated meat by performing 3D printing on the bioink to prepare a three-dimension scaffold material and then inoculating the cells for three-dimensional culture can better simulate the growth environment for cells to develop into muscles in vivo, and the cultivated meat are closer to the tissue morphology of the natural muscles. However, the latter requires the high performance of the bioink. This not only requires the bioink to be printable and edible, but also requires the bioink and its prepared three-dimensional scaffold material to have better biocompatibility, promote adhesion and efficient three-dimensional growth of cells, and have better stability.

Because the polysaccharide and protein materials cannot form a three-dimensional structure with a specified strength to support adhesion of cells, the existing bioink printing materials have poor biocompatibility. Although the biocompatibility of the bioink can be improved through acidation modification of methacrylonitrile, modified materials can be photosensitive materials. Therefore, inedible photoinitiators must be added into the modified materials during printing to form a three-dimensional structure with a specified strength to support the three-dimensional growth of cells, so that the bioink material is inedible and cannot be used for preparing the cultivated meat.

The purpose of the present invention is to prepare a 3D printing bioink for producing the cultivated meat; the bioink is prepared into a scaffold material for three-dimensional growth of cells through 3D printing, and then the cells are inoculated to the scaffold material for the adhesion and three-dimensional growth of the cells. The present invention develops raw materials for bioinks and discovers the raw materials for bioinks that significantly promote the adhesion, proliferation, differentiation and three-dimensional growth of the cells.

Specifically, the present invention provides the following technical solutions:

According to a first aspect, the present invention provides a bioink, where raw materials for preparing the bioink include pectin, glutamine transaminase, a first protein component, and a second protein component;
the first protein component is gelatin and/or collagen; and
the second protein component is protamine.

Glutamine transaminase, also known as transglutaminase (TG enzyme), is a monomeric protein that consists of 331 amino groups with a molecular weight of about 38,000 Da and has an active center. Because it can catalyze the intramolecular and intermolecular covalent cross-linking of protein polypeptides to improve the functional properties of proteins, it is commonly used in packing of meat including ham and sausage as a water retaining agent for the production of low-salt meat products, and there are few reports on glutamine transaminase for the cross-linking of polysaccharides and intermolecular proteins.

According to the present invention, it has been found that the pectin, the first protein component, and the second protein component can be cross-linked under the action of the glutamine transaminase to form a three-dimensional reticular structure with a high strength. This can significantly improve the printability and the biocompatibility of the materials, better support the adhesion and three-dimensional growth of the cells, and causes the cell proliferation to present the spatial three-dimensional growth state.

Preferably, in the raw materials, the mass ratio of the first protein component and the second protein component is 1:(1.6-3). The ratio of the mass of the pectin to the total mass of the first protein component and the second protein component is 1:(0.9-2). The dosage ratios of the first protein component to the second protein component and the pectin to the protein component being controlled in the above ranges is more conducive to improving the printability, biocompatibility and mechanical properties of the bioink.

Preferably, in the raw materials, the glutamine transaminase is 700-800 U/g relative to the total mass of the pectin, the first protein component, and the second protein component. The dosage of the glutamine transaminase being controlled in the range is more conducive to enhancing the stability of the bioink in the culture medium.

Further preferably, the glutamine transaminase is 750-800 U/g relative to the total mass of the pectin, the first protein component, and the second protein component.

In the raw materials, preferably, the pectin has a molecular weight of 250-350 kDa, an esterification degree of greater than 75%, and a mass ratio of neutral sugar to acidic sugar of 1:(2-3); The pectin that satisfies the above performance parameters can improve the adhesion rate of cells to the scaffold material.

On the basis of satisfying the above performance parameters, the source of the pectin is not specially restricted, and the pectin may be a high ester pectin from citrus, beets and apples.

In the raw materials, the gelatin is preferably type A gelatin and/or type A+B gelatin. Other types of gelatin have less effective cross-linking effects or even cannot be cross-linked.

In order to satisfy the edible requirements, the pectin, the first protein component, the second protein component, and the glutamine transaminase used above are all food grade.

Preferably, the pectin, the first protein component, and the second protein component are respectively mixed with the glutamine transaminase by forming an aqueous solution with water as a solvent to obtain a raw material mixed aqueous solution; in the raw material mixed aqueous solution, the concentration (g:mL) of the pectin is 8%-12%, the concentration (g:mL) of the first protein component is 1%-5%, and the concentration (g:mL) of the second protein component is 3%-8%.

In the present invention, it is found through extensive screening and optimization of the concentrations of the components in the raw materials that the concentration of the pectin in the raw materials being higher than the above range leads to the increased viscosity of the bioink, and the printed material is easy to dissolve and collapse in the culture medium and poor stability; the concentration of the pectin being less than the above range leads to the reduced cross-linking degree of the bioink; the concentration of the gelatin in the raw materials being higher than the above range leads to the increased strength of the bioink, which is easy to cause the blockage of the 3D printing head; the concentration of the pectin being lower than the above range leads to the reduced viscosity and printability of the bioink, and even the inability to print; the concentration of silk fibroin in the raw materials being higher than the above range leads to the reduced cross-linking degree of the bioink and the poor stability; and the concentration of silk fibroin being lower than the above range leads to the poor adhesion and growth effects of the cells.

Preferably, in the raw material mixed aqueous solution, the concentration (g:mL) of the glutamine transaminase is 0.5%-1.5%. The concentrations of the glutamine transaminase being higher than the above range lead to the high cross-linking degree of the bioink and the inability to perform 3D printing; the concentrations of the glutamine transaminase being lower than the above range lead to the low cross-linking degree of the bioink and the reduced stability of the bioink; and the printed material cannot be stabilized in the culture medium.

The pectin in the raw materials for preparing the bioink of the present invention can also be used as functional polysaccharides, and plays a role in regulating intestinal flora and promoting cholesterol metabolism in the body. In addition, an appropriate amount of functional polysaccharides and oligosaccharide dietary fibers need to be further added according to the practical application as the functional dietary fibers into the body.

Preferably, the raw materials further include a functional dietary fiber. The mass ratio of the functional dietary fiber to the pectin is 1:(5-10), and preferably 1:(8-10).

The functional dietary fiber includes, but is not limited to, fructoglucan, oligofructose, and galactooligosaccharide.

The above mentioned bioink is a bioink for three-dimensional growth of cells for the preparation of cultivated meat.

According to a second aspect, the present invention further provides a preparation method for the bioink, and the method includes: sequentially adding and well mixing glutamine transaminase, a pectin aqueous solution, a first protein component aqueous solution, and a second protein component aqueous solution into a reaction container, and performing cross-linking on the mixture.

According to the present invention, it is found that the addition sequence of the raw materials has a significant effect on the cross-linking degree and can ensure a suitable cross-linking degree, which significantly increases the printability and the stability of the printed material; the sample addition sequence being changed leads to the high or low cross-linking degree, which directly reduces the printability of the bioink and the stability of the printed material in the culture medium.

The functional dietary fibers are added after the pectin aqueous solution is added and before the gelatin aqueous solution is added.

Preferably, the mixture is sonicated for 15-25 min at 25-35 kHz and incubated for 100-140 min at 40-50° C. before cross-linked. Sonication increases the perturbation of molecules of pectin and protein components in the solution, thereby increasing intermolecular contact and promoting cross-linking.

In some embodiments of the present invention, TG enzyme, a pectin aqueous solution with a concentration of 10%-25%, a gelatin or collagen aqueous solution with a concentration of 2%-25%, and a protamine aqueous solution with a concentration of 10%-40% are sequentially added in a reaction container and well mixed in a vortex manner to obtain the mixture; and the mixture is cross-linked in one step, and the specific conditions are that the mixture is sonicated for 15-25 min at 25-35 kHz and incubated for 100-140 min at 40-50° C.

After the cross-linking is completed, 3D printing can be performed directly, and no photoinitiator is required for printing, that is, the solution obtained through cross-linking is loaded into a syringe for 3D printing, and the printed bioink scaffold material can be irradiated and sterilized to obtain the bioink scaffold material required for the preparation of the cultivated meat.

According to a third aspect, the present invention provides any of the following applications of the bioink, and the applications include:
(1) an application in preparation of a biological scaffold material for three-dimensional growth of cells;
(2) an application in preparation of a cultivated meat; and
(3) an application in in-vitro culture of the cells.

In the application (3), the in-vitro culture of the cells is the in-vitro three-dimensional culture of the cells.

According to a fourth aspect, the present invention provides a biological scaffold material, where the biological scaffold material is prepared by the bioink through 3D printing.

According to a fifth aspect, the present invention provides a preparation method for a biological scaffold material, and the preparation method includes: performing 3D printing on the bioink; and
the 3D printing is performed under the following conditions: an extrusion flow rate of 3.50-4.50 mm³/s, a printing speed of 4.50-5.50 mm/s, and a line spacing of 0.4-0.6 mm.

According to a sixth aspect, the present invention provides a preparation method for a cell cultivated meat, and the method includes: inoculating cells into the biological scaffold material for being cultivated.

The cells include, but are not limited to, myoblast cells, muscle stem cells, muscle satellite cells, muscle precursor cells, etc., and their sources include, but are not limited to, pigs, chickens, cows, sheep, and the like.

The beneficial effect of the present invention is that the bioink material provided by the present invention solves the problems of existing bioinks such as poor biocompatibility, inability to support three-dimensional growth of cells, inedibility, poor printability, and inability to be used in the production and processing of cultivated meat; the bioink is edible without photoinitiators for 3D printing; in addition, it has the good biocompatibility and printability, can better support the three-dimensional growth of cells, has the effect of regulating intestinal flora and promoting cholesterol metabolism, and can be used for proliferation of cells in multiple cell culture systems including cell culture dishes, spinner bottles and bioreactors, and for producing and processing the bio-cultivated meat; therefore it has abroad application prospect in fields of food science, cellular agriculture and biomedicine. Specific advantages include at least the following:
(1) The bioink provided by the present invention can be directly used for 3D printing, without requiring additional inedible photoinitiator. Raw materials are substances allowed to be added to food, which have edible natural properties and can be used for production and processing of bio-cultivated meat;
(2) The reticular structure of the bioink material provided by the present invention can simulate the three-dimensional growth environment in animal cells to the greatest extent, and reproduce the in-vivo environment of cells in vitro, which is conducive to the three-dimensional growth of cells in vitro, enables cells such as muscle stem cells to present a spatial three-dimensional growth state, thereby facilitating directional fusion thereof to form myotube cells;
(3) The bioink material provided by the present invention has excellent biocompatibility, supports cell adhesion and three-dimensional growth and makes proliferation present a state of three-dimensional growth, which effectively promotes large-scale and efficient expansion and culturing of muscle stem cells and other cells, with a cell culture density of up to 5×10⁷ cells/mL;
(4) The bioink material of the present invention can form a stable reticular structure, and will not dissolve or collapse when stabilized in the culture medium at 37° C. for 168 h. It has high stability and facilitates adhesion of cells and delivery of nutriment;
(5) The pectin in the bioink of the present invention is a functional polysaccharide, which, in addition to being used as a bioink material for three-dimensional growth of cells, is also endowed with health effects such as regulating the micro-ecological balance of the intestinal flora of the body, promoting the metabolism of cholesterol of the body, and lowering serum cholesterol, etc., by using thereof in the end-products of bio-cultivated meat;
(6) The materials used for preparation of the bioink of the present invention consist of natural macromolecules with low cost, which can be popularized and applied in the field of cell agriculture on a large scale with low cost, and reduce the production cost of bio-cultivated meat. Pectin can be extracted from the skin residues produced in the processing of fruits and vegetables, and less gelatin is used, which can be obtained from the collagen of bones and skins of pigs, cattle, sheep and other animals. Silk fibroin is a natural macromolecular fibrin extracted from silk; and
(7) The bioink material of the present invention is simple and convenient in preparation, facilitating the popularization and application thereof.

### BRIEF DESCRIPTION OF DRAWINGS

In order to more clearly illustrate the technical solutions in the present invention or prior art, the accompanying drawings that need to be used in the description of the embodiments or prior art will be briefly introduced below. Apparently, the accompanying drawings in the following description are some of the embodiments of the present invention. For persons of ordinary skill in the art, other accompanying drawings can be obtained based on these drawings without any creative efforts.

FIG. 1 shows observation results of cells cultured with a bioink scaffold material in Embodiment 1 of Example 1 of the present invention.

FIG. 2 shows observation results of cells cultured with a bioink scaffold material in Embodiment 2 of Example 1 of the present invention.

FIG. 3 shows observation results of cells cultured with a bioink scaffold material in Embodiment 3 of Example 1 of the present invention.

FIG. 4 shows observation results of cells cultured with a bioink scaffold material in Embodiment 4 of Example 1 of the present invention.

FIG. 5 shows observation results of cells cultured with a bioink scaffold material in Embodiment 5 of Example 1 of the present invention.

FIG. 6 shows observation results of cells cultured with a bioink scaffold material in Embodiment 6 of Example 1 of the present invention.

FIG. 7 shows observation results of cells cultured with a bioink scaffold material in Embodiment 7 of Example 1 of the present invention.

FIG. 8 shows observation results of cells cultured with a bioink scaffold material in Comparative Example 1 of Example 1 of the present invention.

FIG. 9 shows observation results of cells cultured with a bioink scaffold material in Comparative Example 2 of Example 1 of the present invention.

FIG. 10 shows observation results of cells cultured with a bioink scaffold material in Comparative Example 3 of Example 1 of the present invention.

FIG. 11 shows observation results of cells cultured with a bioink scaffold material in Comparative Example 4 of Example 1 of the present invention.

FIG. 12 shows counting statistics of cells cultured with a bioink scaffold material in Example 2 of the present invention.

FIG. 13 shows stability test results of a bioink scaffold material in Example 3 of the present invention.

FIG. 14 and FIG. 15 show rheological feature analysis results of a bioink in Example 4 according to the present invention.

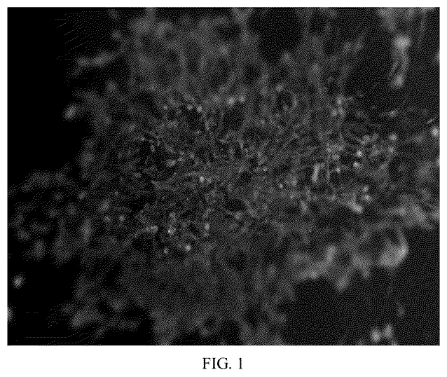

Scales in FIG. 1-FIG. 11 are all 100 μm.

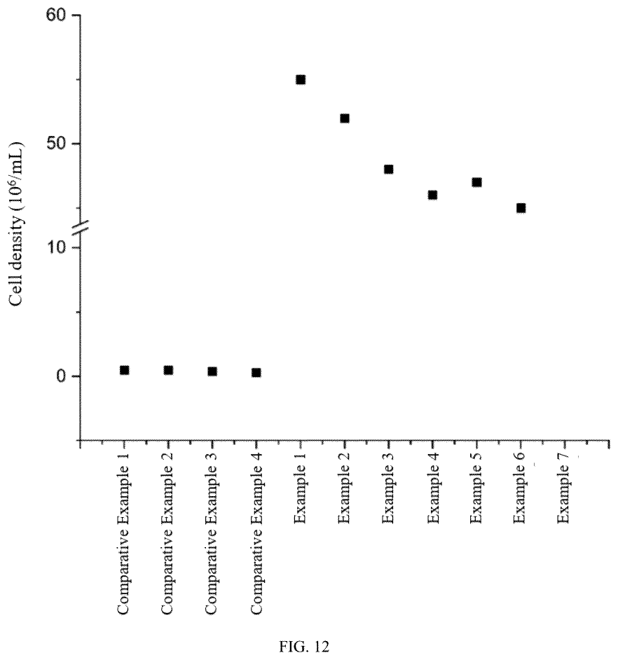

In FIG. 12-FIG. 15, Comparative 1, Comparative 2, Comparative 3 and Comparative 4 respectively represent Comparative Example 1. Comparative Example 2, Comparative Example 3, and Comparative Example 4; Example 1, Example 2, Example 3, Example 4, Example 5, Example 6 and Example 7 respectively represent Embodiment 1, Embodiment 2. Embodiment 3, Embodiment 4, Embodiment 5, Embodiment 6, and Embodiment 7.

### DETAILED DESCRIPTION OF THE EMBODIMENTS

In order to make the purpose, technical solutions and advantages of the present invention clearer, the following will clearly and completely describe the technical solutions of the present invention with reference to the accompanying drawings therein. Apparently, the described embodiments are some of the embodiments of the present invention, instead of all of the embodiments. Based on the embodiments of the present invention, all other embodiments obtained by persons of ordinary skill in the art without any creative efforts fall within the scope of protection of the present invention.

Pectin, glutamine transaminase, gelatin, silk fibroin and other raw materials used in the following embodiments are food grade. The enzymatic activity of the TG enzyme used in the following embodiments is 18,000 U/g to 20,000 U/g.

### Embodiment 1

This embodiment provides a bioink, where raw materials for preparing the bioink include citrus pectin (with a molecular weight of 300 kDa, an esterification degree of 80%, and a mass ratio of neutral sugar to acidic sugar of 1:2), glutamine transaminase, gelatin (type A gelatin) and protamine; in the raw material mixed aqueous solution, the mass ratio of the gelatin to the protamine is 1:3; the ratio of the citrus pectin to the total mass of the gelatin to the protamine is 1:2; the glutamine transaminase is 750 U/g relative to the total mass of the pectin, the gelatin, and the protamine; in the raw material mixed aqueous solution, the concentration of the pectin is 8%, the concentration of the gelatin is 1%, the concentration of the protamine is 3%, and the concentration of the glutamine transaminase is 0.5%.

This embodiment further provides a preparation method for the bioink and a method for preparing a biological scaffold material with the bioink, and the method includes the following steps:
(1) Treatment of raw materials: pectin is prepared into an aqueous solution with a concentration of 16%, gelatin is prepared into an aqueous solution with a concentration of 4%, and protamine is prepared into an aqueous solution with a concentration of 12%;
(2) Mixing of raw materials: 0.5 g of TG enzyme, 50 mL of 16% pectin solution, 25 mL of 4% gelatin solution and 25 mL of 12% protamine solution are added to a beaker sequentially and well mixed in a vortex manner to obtain a mixture;
(3) One-step cross-linking: the mixture is sonicated for 15 min at 25 kHz and incubated for 100 min at 40° C. to obtain a cross-linked solution;
(4) 3D printing: the cross-linked solution is loaded into a syringe, and 3D printing parameters are set as: an extrusion flow rate of 3.50 mm³/s, a printing speed of 4.50 mm/s, and a line spacing of 0.4 mm, and 3D printing is performed to obtain the bioink material; and
(5) Sterilization: the bioink material is subjected to irradiation sterilization.

### Embodiment 2

This embodiment provides a bioink, where raw materials for preparing the bioink include citrus pectin (with a molecular weight of 300 kDa, an esterification degree of 80%, and a mass ratio of neutral sugar to acidic sugar of 1:2), glutamine transaminase, gelatin (type A gelatin) and protamine; in the raw material mixed aqueous solution, the mass ratio of the gelatin to the protamine is 1:1.6; the ratio of the citrus pectin to the total mass of the gelatin to the protamine is 1:0.9; the glutamine transaminase is 800 U/g relative to the total mass of the pectin, the gelatin, and the protamine: in the raw material mixed aqueous solution, the concentration of the pectin is 12%, the concentration of the gelatin is 5%, the concentration of the protamine is 8%, and the concentration of the glutamine transaminase is 1.5%.

This embodiment further provides a preparation method for the bioink and a method for preparing a biological scaffold material with the bioink; and a difference in preparation methods between this embodiment and Embodiment 1 only lies in that: in Step (1) of the preparation method, pectin is prepared into an aqueous solution with a concentration of 24%, gelatin is prepared into an aqueous solution with a concentration of 20%, and protamine is prepared into an aqueous solution with a concentration of 32%.

In Step (2) of the preparation method: 1.5 g of TG enzyme, 50 mL of 24% pectin solution, 25 mL of 20% gelatin solution and 25 mL of 32% protamine solution are added to a beaker sequentially and well mixed in a vortex manner to obtain a mixture.

### Embodiment 3

This embodiment provides a bioink sharing raw materials with that in Embodiment 1.

This embodiment further provides a preparation method for the bioink and a method for preparing a biological scaffold material with the bioink; and a difference in preparation methods between this embodiment and Embodiment 1 only lies in that: in Step (3) of the preparation method, the mixture is sonicated for 25 min at 35 kHz and incubated for 140 min at 50° C. to obtain a cross-linked solution; and in Step (4), 3D printing parameters are set as: an extrusion flow rate of 4.50 mm³/s, a printing speed of 5.50 mm/s, and a line spacing of 0.6 mm.

### Embodiment 4

This embodiment provides a bioink, and a difference in raw materials of the bioink between this embodiment and Embodiment 1 only lies in that: citrus pectin in the preparation raw materials is replaced with apple pectin, and the apple pectin has a molecular weight of 250 kDa, an esterification degree of 78%, and a ratio of neutral sugar to acidic sugar of 1:2.

This embodiment further provides a preparation method for the bioink and a method for preparing a biological scaffold material with the bioink; and a difference in preparation methods between this embodiment and Embodiment 1 only lies in that: the citrus pectin is replaced with the apple pectin.

### Embodiment 5

This embodiment provides a bioink, and a difference in raw materials of the bioink between this embodiment and Embodiment 1 only lies in that: citrus pectin in the preparation raw materials is replaced with beet pectin, and the beet pectin has a molecular weight of 350 kDa, an esterification degree of 85%, and a ratio of neutral sugar to acidic sugar of 1:3.

This embodiment further provides a method for preparing the bioink and a method for preparing a biological scaffold material with the bioink, and a difference in preparation methods between this embodiment and Embodiment 1 only lies in that: the citrus pectin is replaced with the beet pectin.

### Embodiment 6

This embodiment provides a bioink, and a difference in raw materials of the bioink between this embodiment and Embodiment 1 only lies in that: gelatin is replaced with type A+B gelatin.

This embodiment further provides a preparation method for the bioink and a method for preparing a biological scaffold material with the bioink; and a difference in preparation methods between this embodiment and Embodiment 1 only lies in that: the gelatin is replaced with the type A+B gelatin.

### Embodiment 7

This embodiment provides a bioink, and a difference in raw materials of the bioink between this embodiment and Embodiment 1 only lies in that: galactooligosaccharide is further added into the preparation raw materials, the mass ratio of the galactooligosaccharide to the pectin is 1:9; and in the raw material mixed aqueous solution, the concentration of the galactooligosaccharide is 0.9%.

This embodiment further provides a preparation method for the bioink and a method for preparing a biological scaffold material with the bioink; and a difference in preparation methods between this embodiment and Embodiment 1 only lies in that: in Step (2) of the preparation method, 0.9 g galactooligosaccharide is added after the pectin solution is added and before the gelatin solution is added.

### Comparative Example 1

This comparative example provides a bioink, and a difference between this comparative example and Embodiment 1 only lies in that: the citrus pectin of Embodiment 1 is replaced with low-ester citrus pectin, and the low-ester citrus pectin has a molecular weight of 300 kDa, an esterification degree of 40%, and a ratio of neutral sugar to acidic sugar of 1:2.

The comparative example shares the preparation method for the bioink and the method for preparing a biological scaffold material with the bioink with Embodiment 1.

### Comparative Example 2

This comparative example provides a bioink, and a difference between this comparative example and Embodiment 1 only lies in that protamine is removed.

For the preparation method for the bioink and the method for preparing a biological scaffold material with the bioink, a difference between the comparative example and Embodiment 1 only lies in that no protamine is added.

### Comparative Example 3

This comparative example provides a bioink, and a difference between this comparative example and Embodiment 1 only lies in that protamine is replaced with laminin.

For the preparation method for the bioink and the method for preparing a biological scaffold material with the bioink, a difference between the comparative example and Embodiment 1 only lies in that: the protamine is replaced with the laminin.

### Comparative Example 4

This comparative example provides a bioink, and a difference between this comparative example and Embodiment 1 only lies in that citrus pectin is replaced with sodium alginate.

For the preparation method for the bioink and the method for preparing a biological scaffold material with the bioink, a difference between the comparative example and Embodiment 1 only lies in that: the citrus pectin is replaced with the sodium alginate.

### Example 1 Test of the Effect of Bioink Materials on Promoting Three-Dimensional Growth of Cells

The effect of biological scaffold materials prepared by the bioinks in the embodiments and the comparative examples on promoting three-dimensional growth of cells is specifically tested as follows:

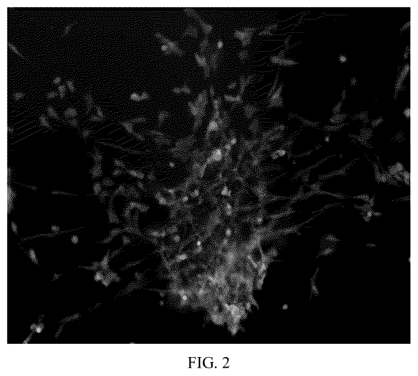

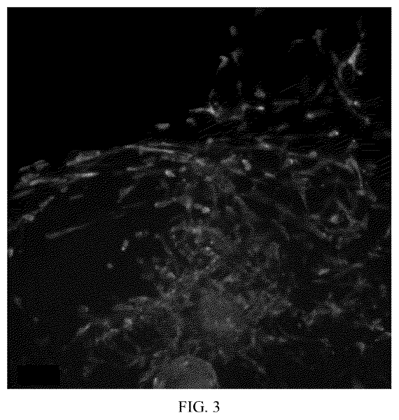

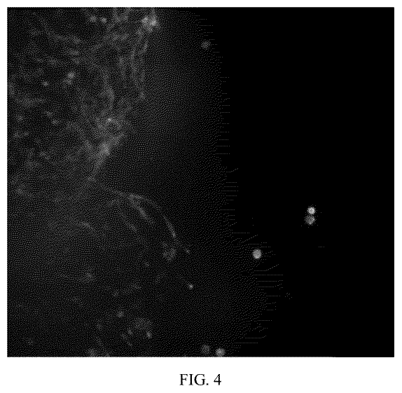

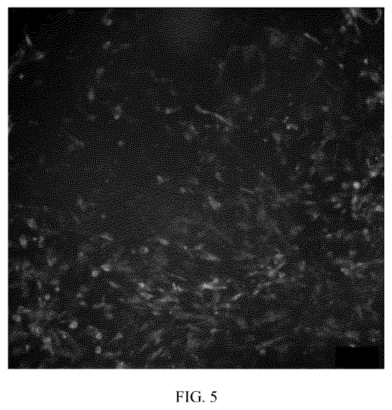

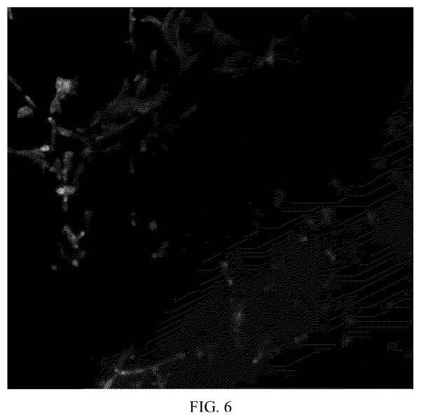

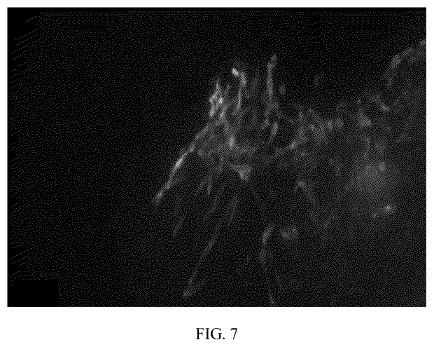

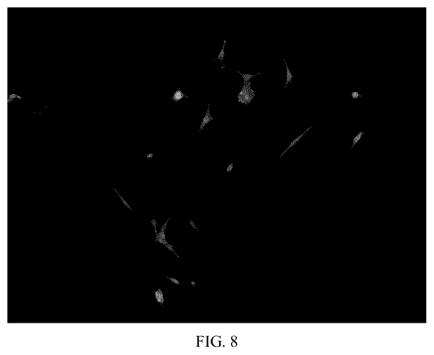

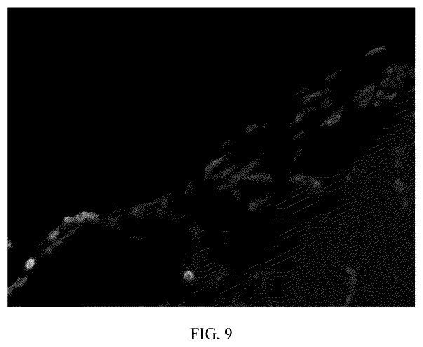

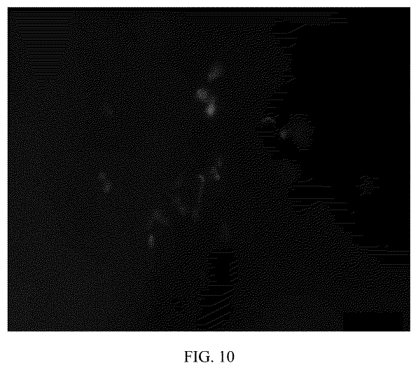

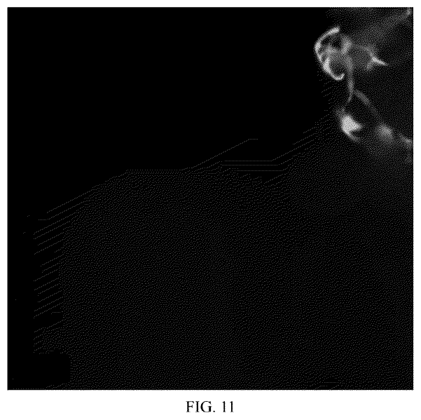

Chicken myoblasts are inoculated into biological scaffold materials prepared by the bioinks in the embodiments and the comparative examples respectively, and cultured for 3 days at a constant temperature of 37° C. with 5% CO₂; cytoskeleton and nucleus are respectively stained by FITC-labeled phalloidin-DAPI and observed for morphology and spatial extension of cells cultured in different groups of biological scaffold materials under a laser confocal high-content analysis platform. Observation results of cells cultured on the biological scaffold materials in Embodiments 1-7 are as shown in FIG. 1, FIG. 2, FIG. 3, FIG. 4, FIG. 5, FIG. 6, and FIG. 7, and observation results of cells cultured on the biological scaffold materials in Comparative Examples 1-4 are as shown in FIG. 8, FIG. 9, FIG. 10, and FIG. 11. The above results show that a large number of cells on the bioink scaffolds in Embodiments 1-7 are in a good growth state and observed for three-dimensional growth at different layers of the bioinks; and cells on the bioink scaffolds in Comparative Examples 1-4 are significantly reduced and in a monolayer growth instead of the three-dimensional growth.

### Example 2 Test of the Effect of Bioink Materials on Promoting Proliferation of Cells

The effect of biological scaffold materials prepared by the bioinks in the embodiments and the comparative examples on promoting proliferation of cells is specifically tested as follows:

After chicken myoblasts are inoculated and cultured for 168 h on the bioink scaffolds respectively and treated with pancreatin, they are resuspended with PBS, stained with Trypan Blue. and counted by an automatic cell counter. Statistical results of cells are as shown in FIG. 12. The results show that an amount of cells cultured on the bioink scaffolds in Embodiments 1-7 is significantly higher than that in Comparative Examples 1-4.

### Example 3 Test of Stability of Bioink Material in Culture Medium

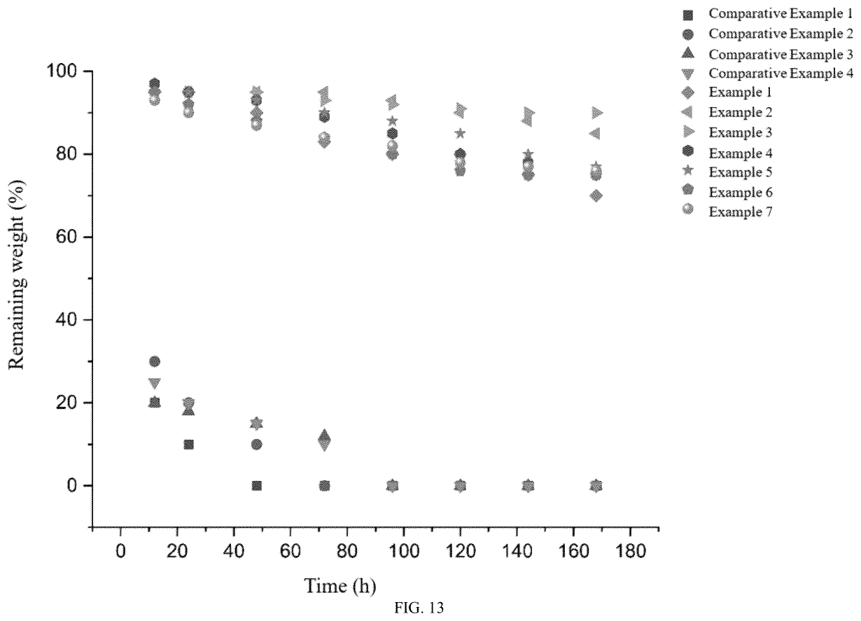

The bioink scaffolds in the embodiments and the comparative examples are weighed under drying conditions, placed in a PBS solution, and incubated in an incubator at 37° C.; and the PBS solution is changed once a day. The cultured bioink scaffolds are removed at set time points, freeze-dried and weighed, and the ratio of the weights of the cultured bioink scaffolds to the weights of the original bioink scaffolds is the remaining weight percentage. Results are as shown in FIG. 13. The results show that the bioink scaffold materials in Embodiments 1-7 can all be stabilized after cultured in the culture medium at 37° C. for 168 h and the remaining weight percentage is 70%-90%, and therefore the bioink scaffold materials have good stability; the bioink materials in Comparative Examples 1-4 are cultured in the culture medium at 37° C. for 12 h and then dissolved, and the remaining weight percentage is 20%-30%; after 96 h, the bioink scaffold materials are all dissolved in the culture medium and have poor stability.

### Example 4 Analysis on Rheological Features of Bioink Material

Rheological features of the bioinks in the embodiments and the comparative examples are specifically tested as follows:

A PP50 probe is used to perform dynamic frequency scanning within the angular frequency of 0.1-100 rad/s under the conditions of a plate spacing of 1 mm, a strain of 1% and a test temperature of 37° C. The samples are left on the platform for 1 min to reach the set temperature before measured. All tests are performed in a linear viscoelasticity region.

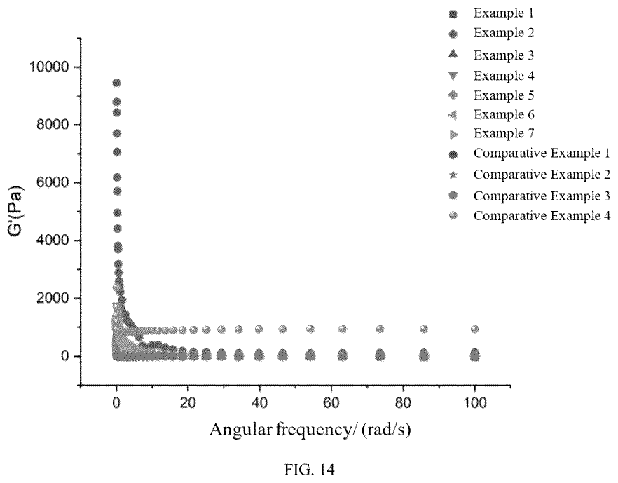

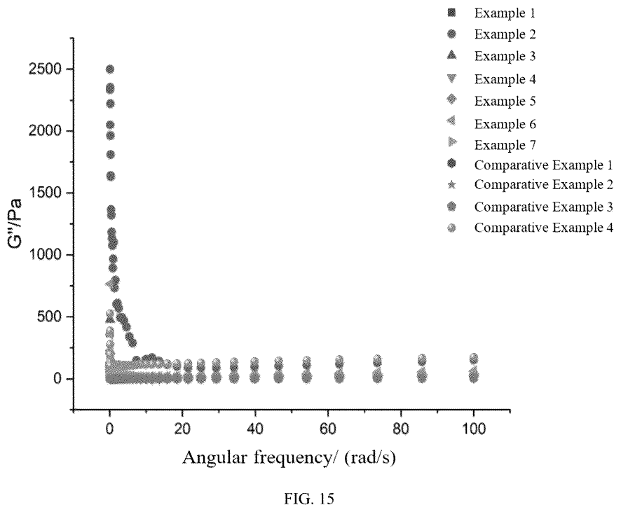

As shown in FIG. 14 and FIG. 15, the angular frequency dependence of the bioinks G′ in Embodiments 1-7 gradually weakens, indicating that the components of the bioinks enhance the gelling property of the material, which helps to maintain the shape of the printed object. In each group, G′ is always greater than G″, indicating that bioinks provide more elasticity and form a stable elastic structure with a strong gel. The elastic structure is able to remain relatively stable under external forces, which facilitates stacking formation of layers during 3D printing deposition. The angular frequency of the bioinks G′ in Comparative Examples 1-4 does not show significant dependence, indicating that the components of the bioinks in the comparative examples do not help to maintain the shape of the printed object.

Finally, it should be noted that the above embodiments are merely intended to illustrate the technical solutions of the present invention, instead of limiting them; although the present invention is detailed with reference to the foregoing embodiments, persons of ordinary skill in the art should understand that it is still possible to make modifications to the technical solutions stated in the foregoing embodiments, or to make equivalent replacements for some of the technical features therein; and such modifications or replacements make the essences of the corresponding technical solutions depart from the spirit and scope of the technical solutions of the embodiments of the present invention.

What is claimed is:

1. A bioink, wherein raw materials for preparing the bioink comprise pectin, glutamine transaminase, a first protein component, and a second protein component;
the first protein component is gelatin and/or collagen;
the second protein component is protamine;
the pectin has a molecular weight of 250-350 kDa, an esterification degree of greater than 75%, and a mass ratio of neutral sugar to acidic sugar of 1:(2-3);
and/or, the gelatin is type A gelatin and/or type A+B gelatin.

2. The bioink according to claim 1, wherein in the raw materials, the mass ratio of the first protein component to the second protein component is 1:(1.6-3);
and/or, the ratio of the mass of the pectin to the total mass of the first protein component and the second protein component is 1:(0.9-2);
and/or, the glutamine transaminase is 700-800 U/g relative to the total mass of the pectin, the first protein component, and the second protein component.

3. The bioink according to claim 1, wherein the pectin, the first protein component, and the second protein component are respectively mixed with the glutamine transaminase by forming an aqueous solution with water as a solvent to obtain a raw material mixed aqueous solution; in the raw material mixed aqueous solution, the concentration of the pectin is 8%-12%, the concentration of the first protein component is 1%-5%, and the concentration of the second protein component is 3%-8%;
and/or, in the raw material mixed aqueous solution, the concentration of the glutamine transaminase is 0.5%-1.5%;
and/or, the raw materials further comprise a functional dietary fiber, and the mass ratio of the functional dietary fiber to the pectin is 1:(5-10).

4. A preparation method for a bioink, comprising: sequentially adding and well mixing glutamine transaminase, a pectin aqueous solution, a first protein component aqueous solution, and a second protein component aqueous solution into a reaction container, and performing cross-linking on the mixture.

5. The preparation method for a bioink according to claim 4, wherein the mixture is sonicated for 15-25 min at 25-35 kHz and incubated for 100-140 min at 40-50° C. before cross-linked.

6. Any of the following applications of the bioink according to claim 1 comprising:
(1) an application in preparation of a biological scaffold material
for three-dimensional growth of cells;
(2) an application in preparation of a cultivated meat; and
(3) an application in in-vitro culture of the cells.

7. The application of the bioink according to claim 6, wherein the biological scaffold material is prepared by the bioink according to claim 1 through 3D printing.

8. The application of the bioink according to claim 7, wherein the preparation method for the biological scaffold material comprises: when the bioink is subjected to 3D printing, the 3D printing is performed under the following conditions: an extrusion flow rate of 3.50-4.50 mm3/s, a printing speed of 4.50-5.50 mm/s, and a line spacing of 0.4-0.6 mm.

9. The application of the bioink according to claim 6, wherein during the preparation of a cultivated meat, cells are inoculated into the biological scaffold material for being cultivated.

10. The application of the bioink according to claim 9, wherein the preparation method for the biological scaffold material comprises: when the bioink is subjected to 3D printing, the 3D printing is performed under the following conditions: an extrusion flow rate of 3.50-4.50 mm3/s, a printing speed of 4.50-5.50 mm/s, and a line spacing of 0.4-0.6 mm.
